(19) United States
(12) Patent Application Publication
(10) Pub. No.: US 2009/0112109 A1
(43) Pub. Date: Apr. 30, 2009

(54) RECONSTRUCTION OF GEOMETRY OF A BODY COMPONENT AND ANALYSIS OF SPATIAL DISTRIBUTION OF ELECTROPHYSIOLOGICAL VALUES

(76) Inventors: Pawel Kuklik, Suprasl (PL); Lukasz Szumowski, Warsaw (PL)

Correspondence Address: FOLEY & LARDNER LLP, 111 HUNTINGTON AVENUE, 26TH FLOOR, BOSTON, MA, 02199-7610, US

(21) Appl. No.: 12/201,727

(22) Filed: Aug. 29, 2008

Related U.S. Application Data
(60) Provisional application No. 60/969,255, filed on Aug. 31, 2007.
(60) Provisional application No. 60/987,175, filed on Nov. 12, 2007.

(51) Int. Cl. A61B 5/0402; A61B 5/04

(52) U.S. Cl. 600/515; 600/300; 600/509; 604/522; 606/32

(57) ABSTRACT

An apparatus, system, and/or method for the reconstruction of geometry of a body component and analysis of spatial distribution of electrophysiological values. The shape of the body component is reconstructed based on coordinates associated with data points (e.g., electrophysiological values). The data points are interpolated to form a value distribution map. The value distribution map corresponds to the shape of the body component. A report (e.g., textual report, graphical report) is generated based on the data points and/or the value distribution map.

### RELATED APPLICATIONS

This application claims the benefit of U.S. Provisional Application No. 60/969,255, filed on Aug. 31, 2007, and U.S. Provisional Application No. 60/987,175, filed on Nov. 12, 2007, which are herein incorporated by reference.

### BACKGROUND

Description of heart muscle electrical activity is essential for the proper treatment of cardiac arrhythmias. Contemporary mapping and ablating systems allow physician to introduce a catheter into the human heart and to measure the position of the electrode in space and, simultaneously, the electrical activity at given position. If enough data points are collected, an approximate reconstruction of the heart chamber geometry is possible together with reconstruction of spatial distribution of electrophysiological values on the surface of the heart. This distribution of electrophysiological parameters is crucial to understand and treatment of life threatening arrhythmias.

### SUMMARY

There are several systems for heart mapping giving a qualitative picture of distribution of the electrophysiological values. The main idea of these systems is to measure local electrogram using a catheter that can be precisely localized in space. If enough endocardial sites are characterized, the three dimensional (3D) geometry of the chamber is reconstructed and analyzed. The analysis of the voltage amplitude and, if possible, of the local activity isochrones during tachycardia allow physicians to recognize the mechanism of arrhythmia and to destroy, by radiofrequent current delivered from an intra cardiac electrode, the substrate crucial for arrhythmia initiation and maintenance.

Unfortunately current mapping systems have limited possibilities in terms of analysis of the received maps, giving only an image of a spatial distribution of the electrophysiological values. The electrophysiological values include local activation time, bipolar voltage (i.e., the signal amplitude which corresponds to the electrical viability of the heart muscle), dominant frequency, signal fragmentation and/or several others values derived from electrogram.

One approach to a quantitative analysis of the distribution of electrophysiological parameters on a body component is a method. The method includes reconstructing a shape of the body component based on coordinates associated with a plurality of data points. The method further includes interpolating the plurality of data points to form a value distribution map corresponding to the shape of the body component and generating a textual report and/or a graphical report based on the plurality of data points and/or the value distribution map.

Another approach to a quantitative analysis of the distribution of electrophysiological parameters on a body component is a computer program product. The computer program product is tangibly embodied in an information carrier. The computer program product includes instructions being operable to cause a data processing apparatus to reconstruct a shape of the body component based on coordinates associated with a plurality of data points. The computer program product further includes instructions operable to cause a data processing apparatus to interpolate the plurality of data points to form value distribution maps corresponding to the shape of the body component and generate a textual report and/or a graphical report based on the plurality of data points and/or the value distribution map.

Another approach to a quantitative analysis of the distribution of electrophysiological parameters on a body component is an apparatus. The apparatus includes a surface reconstruction module, a data interpolation module, and a quantitative analysis module. The surface reconstruction module is for reconstructing a shape of the body component based on coordinates associated with a plurality of data points. The data interpolation module is for interpolating the plurality of data points to form value distribution maps corresponding to the shape of the body component. The quantitative analysis module is for generating a textual report and/or a graphical report based on the plurality of data points and/or the value distribution map.

Another approach to a quantitative analysis of the distribution of electrophysiological parameters on a body component is an apparatus. The apparatus includes a means for reconstructing a shape of the body component based on coordinates associated with a plurality of data points. The apparatus further includes a means for interpolating the plurality of data points to form value distribution maps corresponding to the shape of the body component and a means for generating a textual report and/or a graphical report based on the plurality of data points and/or the value distribution map.

In other examples, any of the approaches above can include one or more of the following features. The data points include electrophysiological data points. The value distribution map includes a 3-dimensional map of spatiotemporal distribution of values associated with the plurality of data points.

In some examples, the coordinates associated with the data points are determined based on electroanatomical information. The plurality of data points are received from a body component sensor.

In other examples, the textual report and/or the graphical report are transmitted to a computing device. The textual report and/or the graphical report are displayed on a display of a computing device.

In some examples, the body component includes a heart, a lung, a liver, a stomach, a muscle, an organ, and/or a tissue. One or more segments of damaged heart muscle areas in the heart associated with a health risk are identified. The coordinates of data points are in 3-dimensional space.

In other examples, the value distribution map is modified based on information associated with the body component sensor. The information associated with the body component sensor includes an electrical potential of the body component. The value distribution map includes a distribution of location activation time map, an electrical viability map, a conduction velocity map, a dominant frequency map, an activation regularity index map, a conduction phase map, and/or an arrhythmogenesis map.

In some examples, inhomogeneity of the body component is determined based on a set of the plurality of data points. Homogeneity of the body component is determined based on statistical properties of a conduction phase map.

In other examples, a conduction phase map is determined based on a conduction heterogeneity index. Homogeneity of the body component is determined based on the conduction phase map.

In some examples, homogeneity of the body component is determined based on minimum, maximum, mean, and/or standard deviation of a set of the plurality of data points and/or the value distribution map. The body component includes a heart muscle and the set of the plurality of data points are associated with one or more ventricles and/or an atria of the heart muscle.

In other examples, a quantifiable risk associated with arrhythmia of the heart muscle is determined. The quantifiable risk is associated with inhomogeneity of the heart muscle and the inhomogeneity being associated with hypertrophic, cardiomyopathy; dilated cardiomyopathy; right ventricle arrhythmogenic cardiomyopathy; ischemic cardiomyopathy; after stem cells implantation in the heart muscle; and/or a genetic disorder.

In some examples, the shape of the body component is automatically divided into a plurality of segments. The textual report and/or the graphical report for each segment in the plurality of segments are generated based on the value distribution map associated with the segment.

In other examples, the shape of the body component is automatically divided into the plurality of segments based on anatomical information associated with the body component. Each segment in the plurality of segments is simultaneously analyzed and compared by an area, a circumference, and/or the data points associated with the plurality of segments.

In some examples, a segment in the plurality of segments is determined to guide a treatment and/or diagnostic procedure. Heart electrical activity is diagnosed. A type and one or more characteristics of an arrhythmia are identified based on the heart electrical activity.

In other examples, an ablation procedure is guided based on an arrhythmogenic effect of the segment. An improvement of a heart muscle after stem cells injection is assessed. A segment associated with the body component is determined based on a relationship between a set of the plurality of data points and/or the value distribution map. The set of the plurality of data points is associated with electrophysiological information.

In some examples, the segment associated with the body component is an arrhythmogenic area of the body component. A geometrical location and spatial distribution of the segment is determined on a surface of the body component.

In other examples, the arrhythmogenic area is associated with an area with low correlation, an area with positive correlation, an area with negative correlation, and/or an area with abrupt change of correlation type. A correlation is determined utilizing a Spearman correlation and/or Pearson correlation.

In some examples, the relationship between the set of the plurality of data points being associated with a potential ablation target. A numerical model of arrhythmias associated with the body component is simulated based on the graphical report. The model includes virtual ablation lines associated with the body component.

In other examples, a quantifiable risk associated with arrhythmia associated with a geometrical feature of the body component is determined. The geometrical feature is associated with a defined voltage and/or a defined conduction velocity.

In some examples, the data points associated with a line are determined. The line is designated based on user parameters and/or geometrical information associated with the body component. An electrophysiological characteristic of the line is determined. The electrophysiological characteristic is displayed which enables a localization of an area associated with arrhythmogenesis.

In other examples, the apparatus includes a surface segmentation module. The surface segmentation module is for automatically dividing the shape of the body component into a plurality of segments.

In some examples, the apparatus includes a map calculation module. The map calculation module is for determining a segment associated with the body component based on a relationship between a set of the plurality of data points. The set of the plurality of data points is associated with electrophysiological information.

In other examples, the apparatus includes a 3-dimensional electroanatomical mapping module. The 3-dimensional electroanatomical mapping module is for determining the coordinates associated with the data points based on electroanatomical information.

The reconstruction of geometry of a heart chamber and analysis of spatial distribution of electrophysiological parameters techniques described herein can provide one or more of the following advantages. An advantage is that the quantitative description of obtained maps and/or derived maps increases the protection of the patients by providing a comprehensive automated examination of the body component and decreases the time for healthcare users to diagnosis health issues.

Another advantage is that the segments/areas of risk can be quickly and efficiently identified based on the electrophysiological values which enable early detection of issues. An additional advantage is that the coordinate system can be utilized to direct a probe to the identified segment/area of risk which enables quick and efficient mitigation of a health risk.

Other aspects and advantages of the present invention will become apparent from the following detailed description, taken in conjunction with the accompanying drawings, illustrating the principles of the invention by way of example only.

### BRIEF DESCRIPTION OF THE DRAWINGS

FIG. 1 illustrates an exemplary mapping apparatus;

FIG. 2 depicts an exemplary flow of data through another exemplary mapping system;

FIG. 3 depicts an exemplary flowchart of electrophysiological values;

FIGS. 4A-D illustrates an exemplary reconstruction of the left ventricle of a heart;

FIGS. 5A-B illustrate a movement of a node i from a edge k during a curvature minimization phase;

FIGS. 6A-B illustrate a voltage interpolation on a reconstructed surface;

FIGS. 7A-D illustrate a measured set of data points and three orthogonal projections of a reconstructed left ventricle of a heart chamber;

FIGS. 8A-B illustrate a map of activation time and conduction velocity of a left ventricle of a heart chamber;

FIGS. 9A-D illustrate maps depicting a mismatch between voltage amplitude and conduction velocity;

FIGS. 10A-B illustrate maps depicting a mismatch between maps of voltage amplitude and conduction velocity;

FIG. 11 depicts an exemplary analysis of a relationship between conduction velocity and bipolar voltage in a left ventricle of a heart chamber;

FIGS. 12A-C illustrate an exemplary analysis of the bipolar voltage and conduction velocity along a line on a heart chamber surface; and

FIGS. 13A-C depict construction of a mathematical model of electrical activity using geometry of a left atria of a heart chamber.

### DETAILED DESCRIPTION

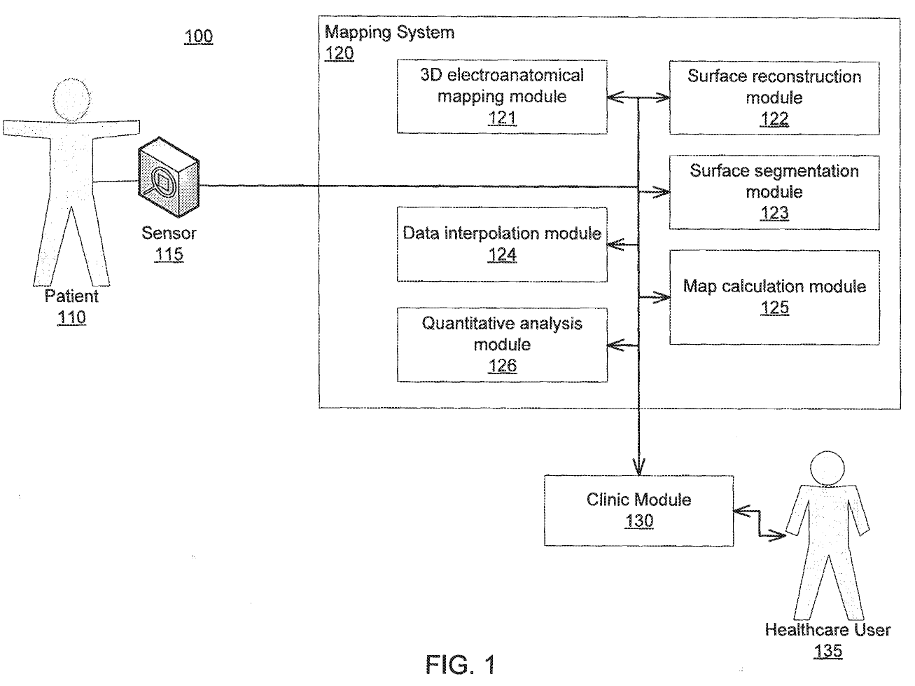

FIG. 1 illustrates an exemplary apparatus 100 for mapping electrophysiological values. The apparatus 100 includes a patient 110, a sensor 115, a mapping system 120, a client module 130, and a healthcare user 135. The mapping system 120 includes a three dimensional (3D) electroanatomical mapping module 121, a surface reconstruction module 122, a surface segmentation module 123, a data interpolation module 124, a map calculation module 125, and a quantitative module 126.

The sensor 115 receives data points associated with a body component of the patient 110 and/or data points associated with other aspects of the patient 110 (e.g., patient's environment, patient's location, etc.). The sensor 115 communicates the plurality of data points to the mapping system 120 and/or the modules associated with the mapping system 120. The sensor 115 can automatically transmit coordinates associated with each data point to the 3-dimensional (3D) electroanatomical mapping module 121. In other examples, a user (not shown) associated with the sensor 115 transmits coordinates associated with each data point to the 3D electroanatomical mapping module 121. In some examples, the 3D electroanatomical mapping module 121 determines the coordinates based on electroanatomical information associated with the data points.

The 3D electroanatomical mapping module 121 communicates the coordinates associated with the plurality of data points to the surface reconstruction module 122. The surface reconstruction module 122 reconstructs a shape of the body component (e.g., heart, lung, etc.) based on the coordinates. The surface reconstruction module 122 communicates the shape of the body component to the surface segmentation module 123.

The surface segmentation module 123 automatically divides the shape of the body component into a plurality of segments. The surface segmentation module 123 can utilize, for example, divide the shape of the body component into the plurality of segments based on anatomical information associated with the body component (e.g., average size of a heart, measured size of a heart, etc.). The surface segmentation module 123 communicates the plurality of segments to the data interpolation module 124.

The data interpolation module 124 interpolates the plurality of data to form a value distribution map corresponding to the shape of the body component. The value distribution map can include, for example, a distribution of location activation time map, a viability map, a conduction velocity map, a dominant frequency map, an activation regularity index map, a conduction phase map, an arrhythmogenesis map, and/or any other type of map associated with electrophysiological parameters. The data interpolation module 124 communicates the value distribution map to the map calculation module 125 and/or the quantitative analysis module 126.

The map calculation module 125 determines a segment associated with the body component based on a relationship between a set of the data points. The set of the data points is associated with electrophysiological information (e.g., current, voltage, etc.). For example, the map calculation module 125 determines the segment associated with the atria of the heart based on the current associated with the atria.

The quantitative analysis module 126 generates a textual report and/or a graphical report based on the value distribution map and/or the data points. The quantitative analysis module 126 can, for example, transmit the textual report and/or the graphical report to a computing device (e.g., the clinic module associated with the healthcare user 135, cell phone, etc.) and/or display the textual report and/or the graphical report on a display of the computing device (e.g., the clinic module associated with the healthcare user 135, desktop computer, laptop computer, cell phone, etc.).

In some examples, the data points are electrophysiological values/data points. The electrophysiological data points can include an electrical property of the body component. The electrical property can include, for example, voltage change, electrical current, and/or any other type of electrical property.

In other examples, the body component is a muscle, an organ, a tissue, and/or any other type of cell associated with an animal (e.g., mammal, reptile, etc.) and/or a human. The organ can include, for example, a heart, a lung, a liver, a stomach, and/or any other type of organ. The body component can be, for example, a heart muscle, and the data points are associated with one or more ventricles and/or an atria of the heart muscle.

In some examples, the value distribution map includes a 3D map of spatiotemporal distribution of values associated with the plurality of data points. The value distribution map can be, for example, a mesh that follows the shape of the body component and includes values that are distributed from the data points. For example, the mesh has real data points—point 0.0, value 0.2; point 0.2, value 0.4—and distributed data points—point 0.1, value 0.3.

In other examples, the value distribution map is modified based on information associated with the body component sensor. The information associated with the body component sensor can include, for example, data points associated with other aspects of the patient 110 (e.g., patient's environment, patient's location, etc.). For example, the other aspects of the patient can include the patient's environment, the patient's location, an electrical potential of the body component, and/or any other type of non-electrophysiogical information.

In some examples, the coordinates are associated with a location of the sensor 115 and/or a probe which is part of the sensor 115. The coordinates can be, for example, in a 3D plane relative to a centralized point (e.g., standard point at the sensor, standard point from a set point on the patient, etc.), relative to each other (e.g., first point at 0,0,0, second point at +1, −1, +3 from the first point, etc.), and/or any other type of coordinate mapping.

In other examples, the map calculation module 125 identifies a segment of damaged heart muscle area in the heart associated with a health risk. The health risk can be an arrhythmia, a myocardial infarction, and/or any other type of health risk associated with the heart. Although the heart and heart muscle area is discussed in this example, other body components can be analyzed to determine segments associated with a health risk (e.g., lung, liver, stomach, etc.).

In some examples, the map calculation module 125 determines a geometrical location and a spatial distribution of the segment on the surface of the body component. The area and/or segment can include, for example, a number of separate areas, a total surface area, a separate surface area, a circumference length, and/or a border zone area (e.g., an area with values in a predefined range).

In other examples, the segment is associated with an arrhythmogenic area. The arrhythmogenic area can be associated with an area with low correlation, an area with positive correlation, an area with negative correlation, and/or an area with abrupt change of correlation type. The map calculation module 125 can determine the correlation utilizing a Spearman correlation, a Pearson correlation, and/or any other type of correlation method.

In some examples, the segment is associated with a muscle area between two other segments. For example, the muscle area has slow conduction properties in comparison to the surrounding segments and therefore creates a substrate for macroreentry—an isthmus.

In other examples, the map calculation module 125 determines inhomogeneity of the body component based on the data points. For example, the map calculation module 125 determines that the heart does not have the same voltage throughout which indicates a certain health risk (e.g., incorrect depolarization, asynchronous depolarization, etc.). The map calculation module 125 can determine homogeneity of the body component based on minimum, maximum, mean, and/or standard deviation of the data points associated with the body component and/or the value distribution map. For example, the homogeneity of the body component is determined by calculating the mean and the standard deviation of the data points and determining if the normal distribution is within the calculated mean and standard deviation (e.g., 99.73% of the data set is within three times the standard deviation on both sides of the mean).

In some examples, the map calculation module 125 determines a conduction phase map based on a conduction heterogeneity index (e.g., pre-determined index, dynamically generated index based on information associated with the patient, etc.). The map calculation module 125 determines homogeneity of the body component based on the conduction phase map.

In other examples, the map calculation module 125 determines a quantifiable risk associated with arrhythmia of the heart muscle and/or any other type of body component associated with arrhythmia. The quantifiable risk can be, for example, associated with inhomogeneity of the heart muscle and/or a geometrical feature of the body component. The inhomogeneity of the heart muscle can be, for example, associated with hypertrophic, cardiomyopathy; dilated cardiomyopathy; right ventricle arrhythmogenic cardiomyopathy; ischemic cardiomyopathy; after stem cells implantation in the heart muscle; a genetically disorder; and/or any other type of inhomogeneity associated with the heart. The geometrical feature can be associated with a defined voltage and/or a defined conduction velocity.

In some examples, the map calculation module 125 simultaneously analyzes and compares an area, a circumference, and/or the data points associated with segments for each segment. For examples, the map calculation module 125 compares the area of the left atrium and the right atrium to determine if the proportions between the areas of the atria is within a specified range.

In other examples, the map calculation module 125 determines a segment in which to guide a treatment and/or diagnostic procedure into and/or through. The segment can be, for example, be associated with an arrhythmogenic. The map calculation module 125 can determine a relationship between data points associate with a potential ablation target, i.e., the target of the ablation procedure. For example, the map calculation module 125 determines the left atrium has abnormal electrical activity and guides a probe (not shown) to the segment associated with the left atrium based on the 3D coordinates associated with the left atrium.

In some examples, the quantitative analysis module 126 diagnoses heart electrical activity and identifies a type and one or more characteristics of an arrhythmia based on the heart electrical activity.

In other examples, the quantitative analysis module 126 enables an ablation procedure to be guided based on an arrhythmogenic effect of the segment. The quantitative analysis module 126 can, for example, assess an improvement of a heart muscle after stem cells injection. The improvement of the heart muscle can be, for the example, the difference of the heart electrical activity before the stem cells injection and after the stem cells injection.

In some examples, the quantitative analysis module 126 generates the textual report and/or the graphical report for each segment based on the value distribution map associated with the segment and/or the data points associated with the segment. For example, the quantitative analysis module 126 generates a graphical report for the left atrium (i.e., first segment), another graphical report for the right atrium (i.e., second segment), a third graphical report for the left ventricle (i.e., third segment), and a fourth graphical report for the right ventricle (i.e., fourth segment).

In other examples, the apparatus 100 is utilized to train healthcare users, e.g., physicians, nurse, other medical staff, etc. The training can be based on numerical arrhythmia models and/or any other type of model associated with the body component.

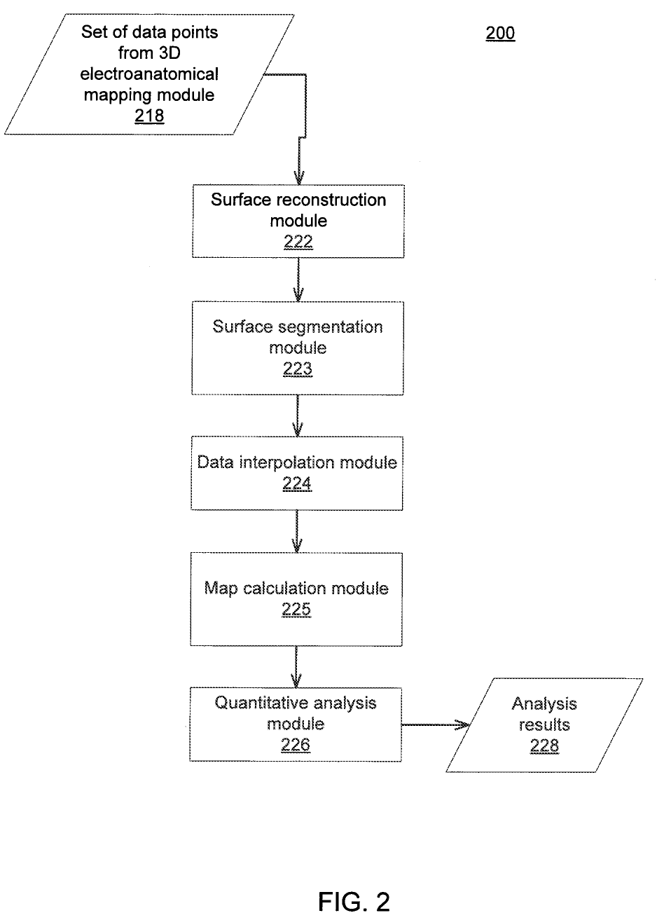

FIG. 2 depicts an exemplary flow of data through another exemplary mapping system 200. A surface reconstruction module 222 receives a set of data points from the 3D electroanatomical mapping module (not shown). The surface reconstruction module 222 reconstructs a shape of the body component based on coordinates. The surface reconstruction module 222 communicates the shape of the body component to a surface segmentation module 223.

The surface segmentation module 223 automatically divides the shape of the body component into a plurality of segments. The surface segmentation module 223 communicates the plurality of segments to a data interpolation module 224. The data interpolation module 224 interpolates the plurality of data to form a value distribution map corresponding to the shape of the body component. The data interpolation module 224 communicates the value distribution map to a map calculation module 225. The map calculation module 225 determines and processes a segment associated with the body component based on a relationship between a set of the data points.

The map calculation module 225 communicates the processed segment and/or the value distribution map to a quantitative analysis module 226. The quantitative analysis module 226 generates analysis results 228 based on the value distribution map and/or the data points.

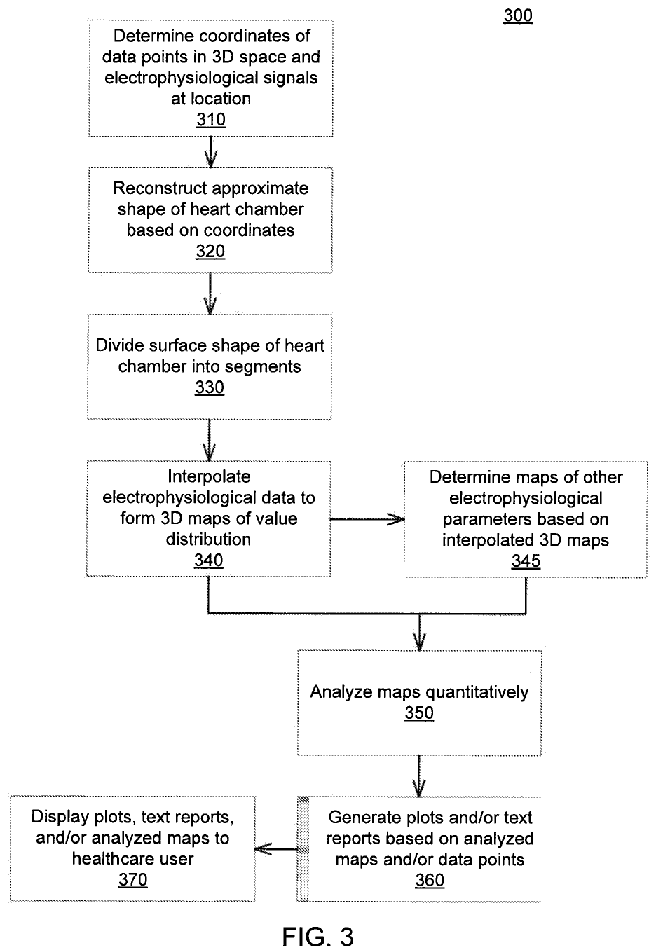

FIG. 3 depicts an exemplary flowchart 300 of electrophysiological parameters through the exemplary apparatus 100 of FIG. 1. The 3D electroanatomical mapping module determines 121 determines (310) coordinates of data points in 3D space and electrophysiological signals at a location. The surface reconstruction module 122 receives a set of data points, the coordinates, and/or the electrophysiological signals from the 3D electroanatomical mapping module 121. The surface reconstruction module 122 reconstructs (320) an approximate shape of a heart chamber on the coordinates. The surface reconstruction module 121 communicates the shape of the heart chamber to the surface segmentation module 123.

The surface segmentation module 123 divides (330) the surface shape of the heart chamber into segments (e.g., left atrium, right atrium, etc.). The surface segmentation module 123 communicates the segments to the data interpolation module 124. The data interpolation module 124 interpolates (340) the plurality of data to form a value distribution map corresponding to the shape of the heart chamber. The data interpolation module 124 communicates the value distribution map to a map calculation module 125 and the quantitative analysis module 126. The map calculation module 125 determines (345) maps of other electrophysiological parameters based on the interpolated 3D maps.

The map calculation module 125 communicates the maps of other electrophysiological parameters to the quantitative analysis module 126. The quantitative analysis module 126 quantitatively analyzes (350) the value distribution map and/or the maps of other electrophysiological parameters. The quantitative analysis module 126 generates (360) plots and/or text reports based on the analyzed maps (e.g., graphical representation of the current on the surface of the heart chamber, graphical representation of the current on the surface of the heart chamber each second over sixty seconds, etc.) and/or the data points. The quantitative analysis module 126 communicates the plots and/or the text reports to the clinic module 130. The clinic module 130 displays (370) the plots, the text reports, and/or the analyzed maps to the healthcare user 135.

Although the reconstruction techniques are described above, the apparatus 100 can utilize any type of technique and/or algorithm to reconstruct electrophysiological maps of body components. The description of the examples of the apparatus 100 include various energy functional approaches that can be utilized and any other type of energy functional approach can be utilized in the exemplary apparatus 100.

### Reconstruction

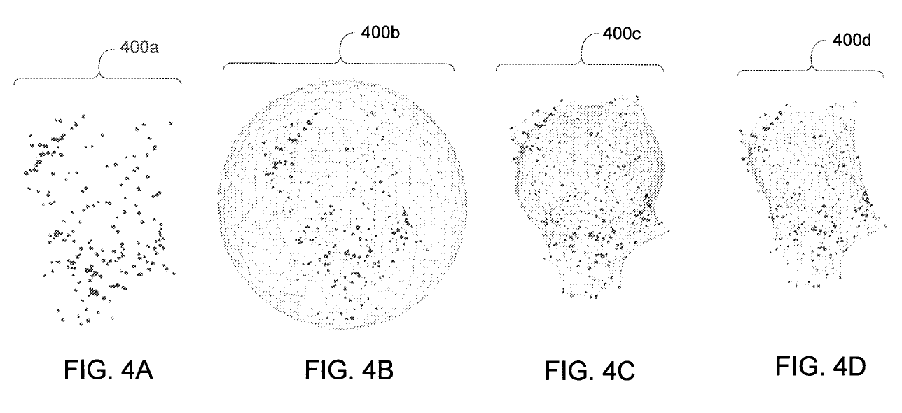

FIGS. 4A-D are an example of reconstruction of the left ventricle of a heart by the surface reconstruction module 122 of FIG. 1. A set of data points 400a is encompassed by a spherical surface 400b. An intermediate phase 400c is shown between the spherical surface 400b and the final result 400d. The final result 400d of the reconstruction is illustrated.

In some examples, the initial surface 400b enclosing the set of the data points 400a is successively deformed until its distance to set of data points is minimized (i.e., the intermediate phase 400c). Triangulated surface representation can be, for example, used because of its simplicity and the small computational effort required during the analysis. The surface can be composed of a set of triangles and generated by the triangulation of the set of points 400a uniformly distributed on a sphere 400b of given radius enclosing the data points as illustrated in FIGS. 4A-D. The deformation process can be directed by two components: the attraction of the surface by the data points and the avoidance of a high curvature of the resultant surface. In other examples, a surface refining process is conducted to avoid overlapping of the surface nodes. The reconstruction stops when the surface is not significantly deformed in one step (i.e., the final result 400d).

In other examples, each step of a surface node movement, is composed of two terms:

δrᵢ=(δaᵢ+δcᵢ)δsᵢ (Equation 1)

where i is the node index, δaᵢ is the movement due to the attraction of a node by the data points, and δcᵢ is due to the minimization of the curvature of the surface. δsᵢ is an adaptive spatial step defined as

$$δ {s}_{i} = \frac{0.001}{N} {\sum }_{j ∈ {A}_{i}} \vert {r}_{i} - {r}_{j} \vert \qquad ( Equation 2 )$$

where Aᵢ is set of neighbors of node i and N is their number. This value of is greater in the initial phase of the reconstruction and decreases as the nodes approach each other. δaᵢ is defined as:

δaᵢ=−CₐgradDᵢ (Equation 3)

where Cₐ is a parameter determining the attraction strength, while the distance function

$${D}_{i} = {\sum }_{j ∈ {S}_{i}} \vert {r}_{i} - {p}_{j} \vert \qquad ( Equation 4 )$$

is the sum of the distances from the data point i in the node neighborhood Sᵢ, which is a sphere at rᵢ with a fixed radius. Limitation of the range of the distance function makes computation faster and prevents an outlying node from being attracted by the more dense areas of the set of the data points. This makes the reconstruction algorithm advantageously less vulnerable: to a nonuniform spatial distribution of data points.

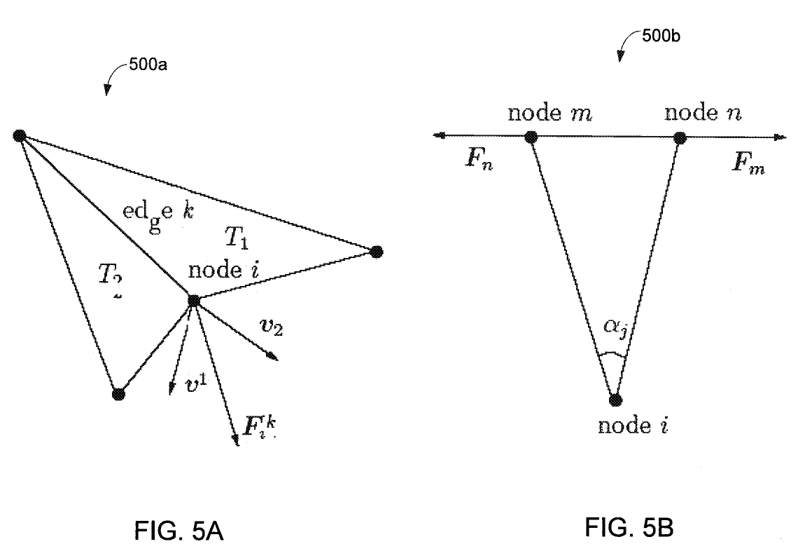

FIGS. 5A-B illustrate the movement of the node i from the edge k during curvature minimization phase. Vector v₁ (v₂) is perpendicular to triangle T₁ (T₂) and its length is equal to the surface of T₁ (T₂) as illustrated in the diagram 500a of FIG. 5A. F_{i,k} is proportional to the sum of v₁ and v₂. The contribution to the movement of the m and n nodes from angle αⱼ due to a curvature minimization as shown in equation 5 is illustrated in the diagram 500b of FIG. 5B. The curvature minimization can move the nodes so that the angle between each pair of triangles sharing a common edge approaches 180 degrees. Thus, the second term, cᵢ is computed as a sum of contributions from the “flattening” of the surface formed by a pair of triangles with a common edge

$$δ {c}_{i} = {C}_{c} {\sum }_{k ∈ {K}_{i}} {F}_{i , k} \qquad ( Equation 5 )$$

where the parameter Cc determines the effect of the movement due to the curvature minimization, Kᵢ is a set of indexes of the edges having common node i. F_{i,k} is the contribution to the movement of the node i due to the processing of the edge k (FIG. 5A).

$$\begin{matrix}{F}_{ik} = ( cos α - 1 ) ( {v}_{1} + {v}_{2} ) & ( Equation 6 ) \\ cos α = \frac{{v}_{1} \cdot {v}_{2}}{\vert {v}_{1} \vert \vert {v}_{1} \vert} & ( Equation 7 )\end{matrix}$$

where v₁ and v₂ are vectors perpendicular to the triangles sharing a common node of a length equal to the area of the given triangle (v then is a vector product of two triangle edges).

During deformation, the nodes are moved toward the data points. If a node i is closer than a certain distance dᵢ to one of the data points, it is pinned so that data point and this node is excluded from further computations. In this example,

$${d}_{i} = \frac{1}{2} 〈 {r}_{i} 〉 \qquad ( Equation 8 )$$

where <rᵢ> is the mean distance from the node i to its neighbors. This procedure prevents a data point from attracting too many nodes and resulting in an unlimited growth of the local node density.

The interplay between the attraction term (equation 3) and the curvature minimization term (equation 5), reflected in the values of parameters Cₐ and C_{c}, can determine the final shape of the surface. In some examples, the values of those parameters are advantageously set empirically in such a way that the resultant surface is smooth and reconstructs the set of data points as exactly as possible. The parameters can be, for example, set to Cₐ=6×10⁴ and C_{c}=15. However, when C_{c} is too small relative to Cₐ, there is a possibility that the areas of high curvature may not be flattened by the curvature minimization term, leading in some cases to a numerical instability. On the other hand, a too high value of C_{c} can cause the curvature minimization term to be greater than the attraction term, preventing the formation of proper curvatures. Although certain behaviors are generally observed, the parameters can be modified and/or set based on the needs and/or goals of the system.

In other examples, the nodes may move too close to each other during the reconstruction process, which may result in the nodes overlapping and the algorithm crashing. In order to avoid such a situation, a refining procedure can be inserted in every reconstruction step. During the refining procedure, each node can be moved by the vector:

δrᵢ=(δdᵢ+δfᵢ)δsᵢ (Equation 9)

where δdᵢ is the movement due to the equalization of the distance from node i to the closest neighbors and δfᵢ is the movement due to the equalization of the angles formed by the edges coming out from node i (see below). δsi is the adaptive spatial step defined in equation 2.

$$δ {d}_{i} = - {C}_{d} {\sum }_{j ∈ {S}_{i}} ( {r}_{j} - {r}_{i} ) ( 1 - \frac{〈 r 〉}{\vert {r}_{j} - {r}_{i} \vert} ) \qquad ( Equation 10 )$$

where C_{d} is equal 35, Sᵢ is a set of the neighbors of the node i and <r> is the mean distance from node i to the closest neighbors:

$$〈 r 〉 = \frac{1}{N} {\sum }_{j ∈ {S}_{1}} \vert {r}_{j} - {r}_{i} \vert \qquad ( Equation 11 )$$

where N is the number of the node i neighbors.

In some examples, the movement due to the equalization of the angles formed by the edges for a given node is not explicitly computed. Instead, each angle is processed, giving two contributions to the motion of the nodes lying at the ends of the edges forming the given angle αᵢ as shown in FIG. 3B,

$$\begin{matrix}{F}_{m} = {C}_{f} ( \frac{2 π}{N} - {α}_{j} ) \frac{{r}_{m} - {r}_{n}}{\vert {r}_{m} - {r}_{n} \vert} & ( Equation 12 ) \\ {F}_{n} = {C}_{f} ( \frac{2 π}{N} - {α}_{j} ) \frac{{r}_{n} - {r}_{m}}{\vert {r}_{n} - {r}_{m} \vert} & ( Equation 13 )\end{matrix}$$

where C_{f} is equal to 150. N is the number of neighbors of the angle αᵢ vertex.

For example, the reconstruction processing begins at the initial condition 400a of FIG. 4A, i.e., a triangulated sphere enclosing the set of data points. The surface reconstruction module 122 stops when no node is moved significantly in one step. The main stages of the reconstruction processing are depicted the diagrams of FIGS. 4A-D.

In other examples, the initial surface in reconstruction phase is a sphere, and therefore the reconstructed surface is a closed manifold. The introduction of anatomical holes (e.g., valves, openings of blood vessels, etc.) is accomplished by pointing three nodes located on the circumference of a given opening. The position of those nodes is determined during ablation when the healthcare user (e.g., doctor, nurse, technician, etc.) begins a procedure by the localization of all chamber openings. Given three nodes, a circular region can be removed from the surface assuming that nodes were located on circumference of the opening.

### Interpolation

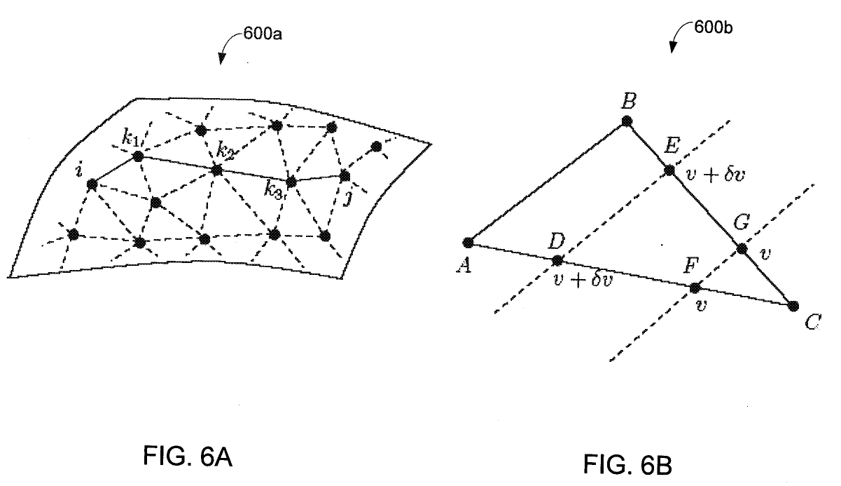

FIGS. 6A-B illustrate a voltage interpolation on a reconstructed surface. The surface 600a illustrates a geodetic line connecting node i and j. In this example, G_{i,j}={i, k1, k2, k3, j}. The distance between the nodes i and j is di,j=|rₖ₁−rᵢ|+|rₖ₂−rₖ₁|+|rₖ₃−rₖ₂|+|rⱼ−rₖ₃|. The surface 600b illustrates the calculation of the area of the surface on which the voltage is in the range (v, v+δv), using the information that lines of constant voltage are straight. Thus, in this example, the area sought is equal to the difference of the areas of the triangles CDE and CFG.

In some examples, the interpolation of the voltage measured at data points to all nodes of the reconstructed surface is analyzed by the surface reconstruction module 122. The linear interpolation can be utilized when there is a lack of additional information about voltage variation along the surface of the body component (i.e., ventricle). Before the interpolation, a metric of the surface is calculated. For example, a set of geodetic lines G_{i,j} joining each pair of nodes is calculated. Each element of G_{i,j} gives a set of nodes connecting nodes i and j by the shortest distance, equal to di,j (FIG. 6A). G_{i,j} and d_{i,j} can be determined using the fast marching method and/or any other path method.

The surface 600b of FIG. 6B illustrates exemplary steps for the interpolation of values. The first step of the interpolation can be the projection of the values from each data point to the closest node. If two or more data points share the same node, an average can be computed. The second stage of the interpolation can be the iteration of four sub-steps:

(i) Find a node with the voltage already assigned and label the node A.

(ii) Find the node closest to node A without an assigned voltage and label the node B.

(iii) Find the node closest to node A where the voltage has been assigned and B lies on a geodetic line G_{A,C}. Call the node C.

(iv) Perform a walk along G_{A,C} assigning each node (including node B) a voltage, assuming linear interpolation:

$${v}_{k} = \frac{{v}_{C} - {v}_{A}}{{d}_{A , C}} {d}_{k , A} + {v}_{A} \qquad ( Equation 14 )$$

where vₖ is the computed voltage of node k lying on the geodetic line G_{A,C} between the nodes A and C. v_{A} and v_{C} are the voltages of nodes A and C respectively. d_{k,A} is the distance between the nodes k and A. The above procedure is iterated as long as all three nodes A, B and C are found in a single step.

If there is a single node that does not lie on any of the geodetic lines, sub-step (iii) fails. The voltage of such an isolated node can be, for example, determined as an average of the voltages of its neighbors.

The last step is voltage interpolation inside a single triangle, with the range of voltages given at its vertices. The voltage of point P in the triangle is computed by determining its barycentric coordinates in a triangle plane

v_{P}=avᵢ+bvⱼ+cvₖ (Equation 15)

where (a, b, c) are barycentric coordinates of point P, and vᵢ, vⱼ and vₖ are voltages of triangle vertices i, j and k.

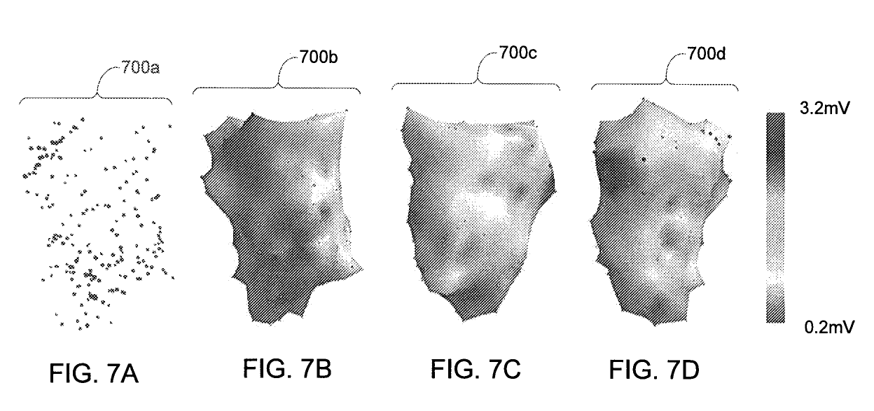

Result of the value interpolation is presented in FIGS. 7A-D. FIGS. 7A-D illustrate a measured set of data points 700a and three orthogonal projections 700b, 700c, and 700d of a reconstructed left ventricle of a heart chamber. In this example, the apex of the heart is at the bottom. The orthogonal projections 700b, 700c, and 700d illustrate the voltage across the surface of the heart.

### Maps

The map calculation module 125 can calculate a conduction velocity (CV) map based on a map of the local activation times (LAT). At each data point on the atria, a surface gradient (spatial derivative) of LAT is calculated. The CV is equal to an inverted absolute value of the gradient vector at given point:

$$C V ( {x}_{0} , {y}_{0} ) = \frac{1}{\vert ∇ L A T ( x , y ) \vert} {\vert}_{{x = {x}_{0}}_{y = {y}_{0}}} \qquad ( Equation 16 )$$

where CV(x₀,y₀) is local conduction velocity at given data point (x₀,y₀) in local coordinate system (x,y). LAT(x,y) denotes local map of activation times. All data points which are extremes of LAT value (beginning and the end of activation) can be removed.

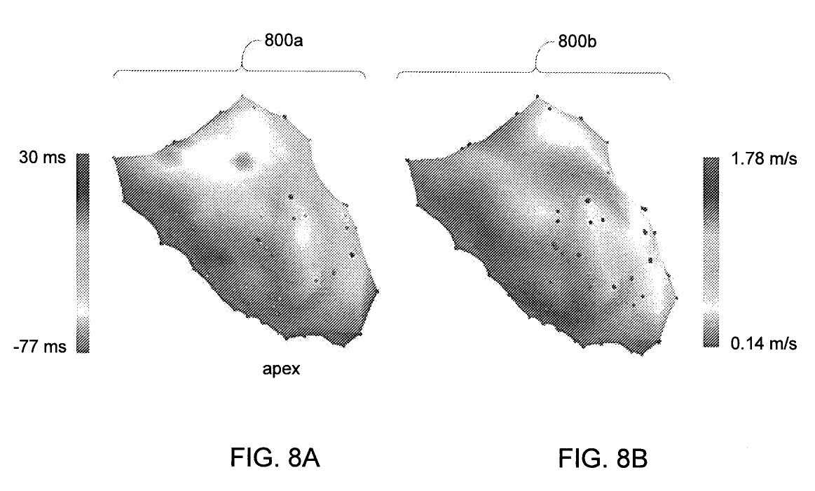

In some examples, a spatial derivative can be replaced in a case of discrete surface with approximation:

$$C {V}_{i} = \frac{{d}_{k , l}}{{t}_{k} - {t}_{l}} \qquad ( Equation 17 )$$

where CVᵢ is a conduction velocity at a node i. k is an index of the neighbor node with the greatest LAT and l with the minimal one (counted in the closest neighborhood of the node i). d_{k,l} is a distance between nodes k and l. tₖ and tₗ are their LAT values. Such calculated CV values can then interpolated on whole surface as illustrated by the map of activation time 800a and the map of conduction velocity 800b of FIGS. 8A-B, respectively. FIGS. 8A-B illustrate a map of activation time 800a and conduction velocity 800b, respectively, of a left ventricle of a heart chamber. The maps 800a and 800b illustrate an anterior view with the apex at the bottom.

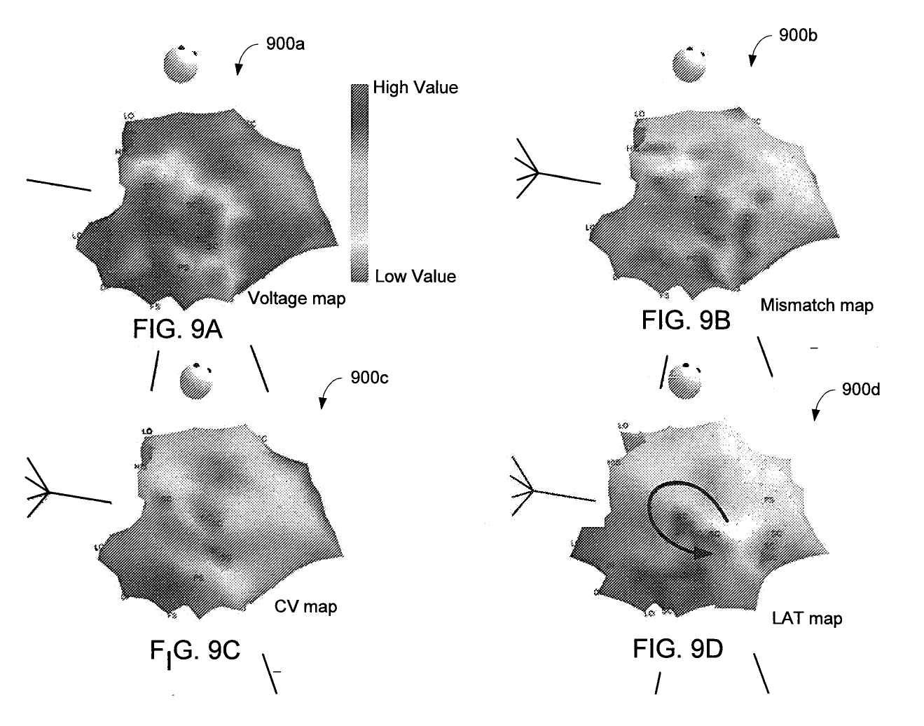

FIGS. 9A-D illustrate maps depicting a mismatch between voltage amplitude and conduction velocity. FIGS. 9A and 9C illustrate maps of mismatch between a map of voltage amplitude 900a and a map of conduction velocity 900c. FIGS. 9D and 9B illustrate a local activation map 900d with a reentry loop (one of the mechanisms of ventricular tachycardia) in an area corresponding. with high spatial variability on the mismatch map 900b, respectively.

In some examples, the calculation of 2-value mismatch map and mismatch gradient map is performed in the following way. Interdependencies between different electrophysiological values play significant role in understanding and treatment of cardiac arrhythmias. For example, in general, low bipolar voltage corresponds with low conduction velocity. Locations where this relation reverses (mismatch areas) can be responsible for maintenance of arrhythmia. The map calculation module 125 calculates a map of mismatch between any two selected electrophysiological values as difference between values after normalization (e.g., average values are zero and standard deviations equal to one). As discussed above, an example of the mismatch map 900b between conduction velocity and bipolar voltage is illustrated in FIG. 9B.

In some examples, the local activation map 900d illustrates a reentry loop (one of the mechanisms of ventricular tachycardia) in an area corresponding with high spatial variability on the mismatch map 900b. The map of mismatch can be used to calculate map of mismatch gradient (spatial derivative of mismatch map). The map of mismatch gradient can be used to assess degree of spatial variability of the mismatch map.

In some examples, the quantitative description of electrophysiological values include:
calculation of the area in which given value is in specified range;
assessment of the number of scars defined as the number of distinct areas with value less than defined threshold;
calculation circumferences of scars and the detection of scars which are close to each other; and/or
calculation of minimum, maximum, mean and/or standard deviation of values on whole surface and/or segments.

In other examples, the quantitative analysis module 126 utilizes prognostic factors and/or quantitative hypotheses to determine a distribution of viability, conduction velocity, and/or other electrophysiological values. The quantitative analysis module 126 can utilize the prognostic factors and/or quantitative hypotheses to determine arrhythmogenesis.

In some examples, the quantitative analysis module 126 localizes an area between segments based on electrophysiological value. For example, the quantitative analysis module 126 determines a localized area and/or line between the left atria and the right atria with a pre-defined and/or dynamically generated voltage. The clinic module 130 can display the electrophysiological characteristics associated with the localized area and/or the line.

In other examples, the quantitative analysis module 126 determines a relationship between two or more electrophysiological values to identify a health risk. For example, the quantitative analysis module 126 determines a relationship between voltage and electrical activation velocity to a potential ablation target in a patient. The identified health risk can include, for examples, a cardioverter-defibrillator intervention, an implanted cardioverter-defibrillator (ICD) as prevention of ICD intervention, and/or other cardiovascular abnormalities. In some examples, the quantitative analysis module 126 determines the relationship between two or more electrophysiological values to determine susceptible to inducing arrhythmias and/or mapping arrhythmias during an electrophysiological study and/or ablation.

In other examples, the quantitative analysis module 126 analyzes electrophysiological values along a line of the body component. The line can be pre-defined (e.g., line between right atria and left atria), dynamically generated (e.g., line between high voltage and low voltage segments of the body component), based on user parameters, and/or geometrical information associated with the body part. The lines can be any type of delineation between segments and/or parts of the body component (e.g., isthmus, line around scars, etc.).

In other examples, the area of the muscle surface in which the voltage is in the range (v, v+δv) is calculated using the fact that due to equation 15 the lines of constant voltage within a triangle are straight. The area inside a single triangle can be calculated as the area of a quadrangle (if there are two lines, v=const and v+δv=const) and/or a triangle (for one line, v=const. or δv+=const) as illustrated in FIG. 6B.

In some examples, the calculation of the area as described above is used to compute the circumferences of scars by calculating the length of the lines of constant value. A scar can be, for example, identified as a group of nodes with a value less than defined threshold. The scar can be detected by performing a walk through all neighboring nodes beginning at the initial, untagged node. Scar detection can be stopped when all nodes with value less than defined threshold have been tagged. In other examples, the area of the scar is computed by making a histogram of the surface corresponding to the value in defined range with the restriction to triangles with nodes belonging to this scar. Analogously, with this restriction, the circumference of a given scar can be calculated. Table 11 illustrates an analysis of bipolar viability of the LV of a patient suffering from ventricular tachycardia, i.e., a textual report.

TABLE 1

| | | |
|---|---|---|
| Data points number | 107 | |
| Chamber surface | 23485 | mm² |
| Scar area | 4969 | mm² |
| Intermediate area | 7274 | mm² |
| Circumference of scars bigger than 100 mm² | 486 | mm |
| Healthy muscle circumference | 437 | mm |
| Healthy area | 11242 | mm² |
| Number of scars bigger than 100 mm² | 2 | |
| Total area of scars bigger than 100 mm² | 5074 | mm² |
| Chamber volume | 148 | cm³ |
| X dimension | 121 | mm |
| Y dimension | 79 | mm |
| Z dimension | 71 | mm |

### Segmentation

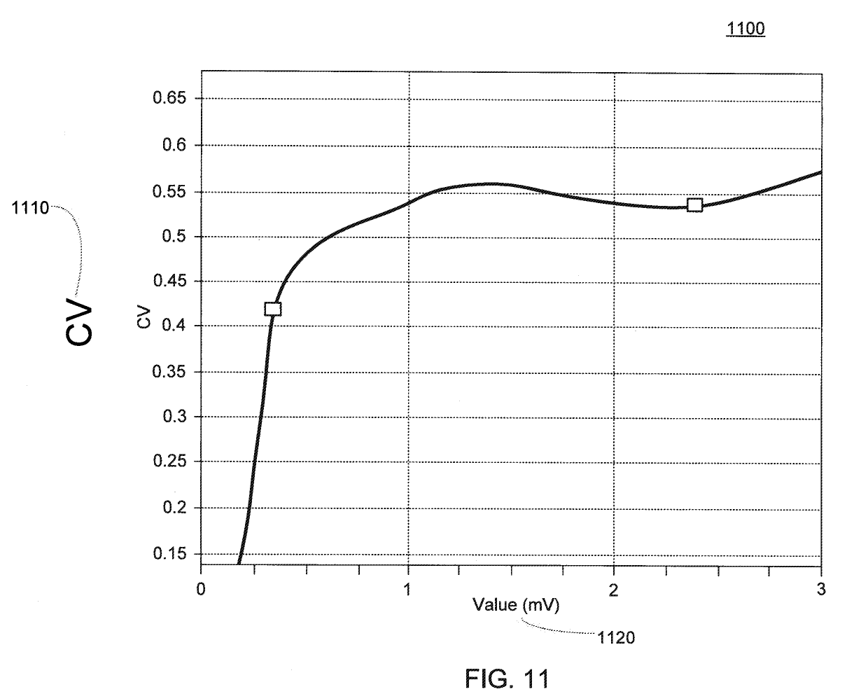

FIG. 11 depicts an exemplary analysis of the relationship chart 1100 between conduction velocity (CV) 1110 and bipolar voltage 1120 in a left ventricle of a heart chamber. The surface segmentation module 123 can identify characteristic “breaking points” (i.e., the points in the line on the chart 1100) denoting change in trend of relationship via the chart 1100.

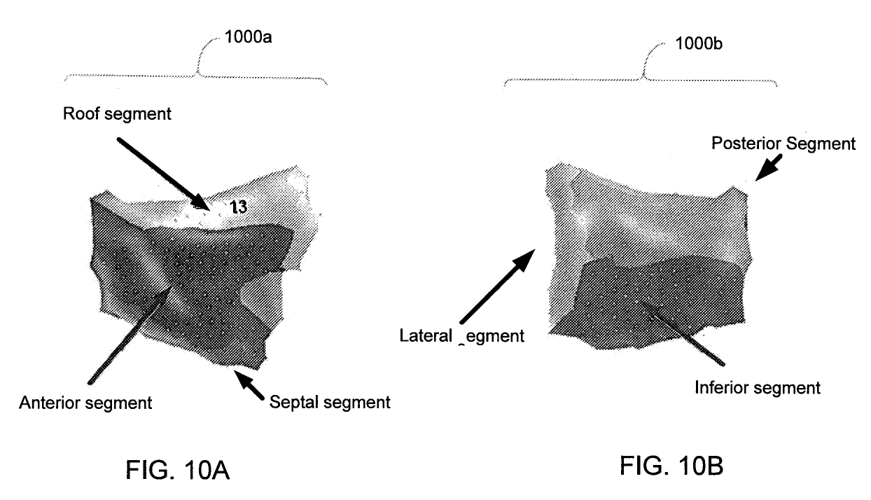

In general, anatomists have divided the surface of the heart chambers into several distinct areas according to their geometrical and functional features. This division was naturally adopted by cardiology and is used, e.g., in description of the distribution of the heart electrophysiological values. The surface segmentation module 123 divides the surface of the chamber into several distinct segments. The map calculation module 125 and/or the quantitative analysis module 126 can perform statistical analysis of values in the segments. FIGS. 10A-B illustrate maps depicting segmentation of the left atria posterior wall 1000a and quantitative analysis of the bipolar voltage 1000b.

In some examples, the electrophysiological values in the data points are mutually related with each other in form that could be not assessed from visual comparison of 3D maps. The quantitative analysis module 126 creates a plot showing functional relationship between any two values on whole surface and/or chosen segment.

In other examples, a moving average is used to remove noise. Any global changes in trends can be, for example, detected by “breaking points” algorithm.

In some examples, the quantitative analysis module 126 allows a user, e.g., healthcare user 135, to introduce a line on chamber surface and analyze values along this line. The analysis can, for example, include:
creation of graphical plot which may be used to visually inspect variability of values along introduced line; and/or
calculation of linear correlation between any two values.

### Analysis

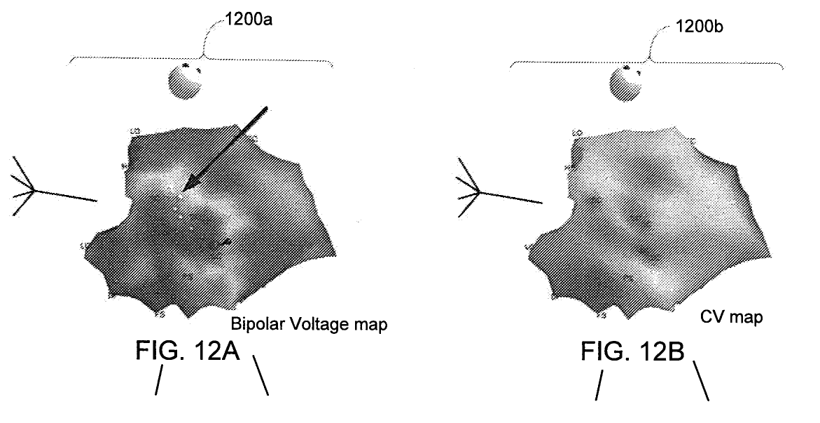

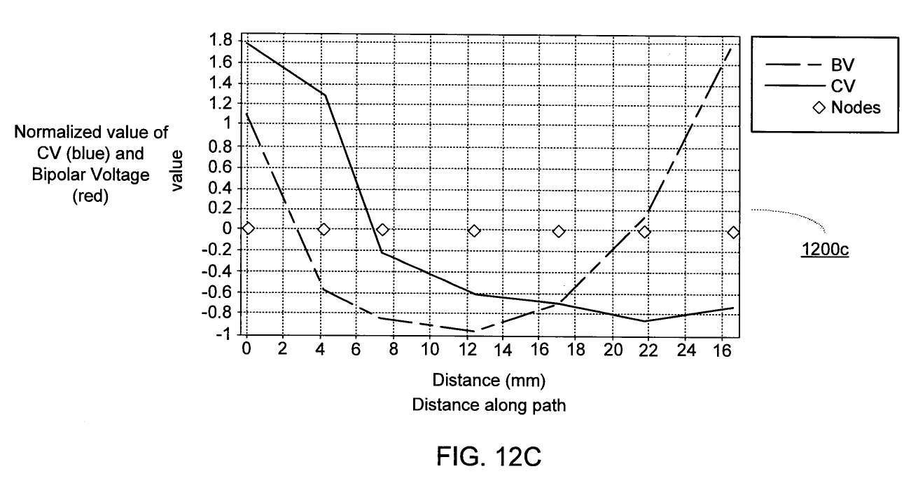

FIGS. 12A-C illustrate an exemplary analysis of a bipolar voltage map 1200a and a conduction velocity map (CV) 1200b along a line on chamber surface as illustrated by the chart 1200c.

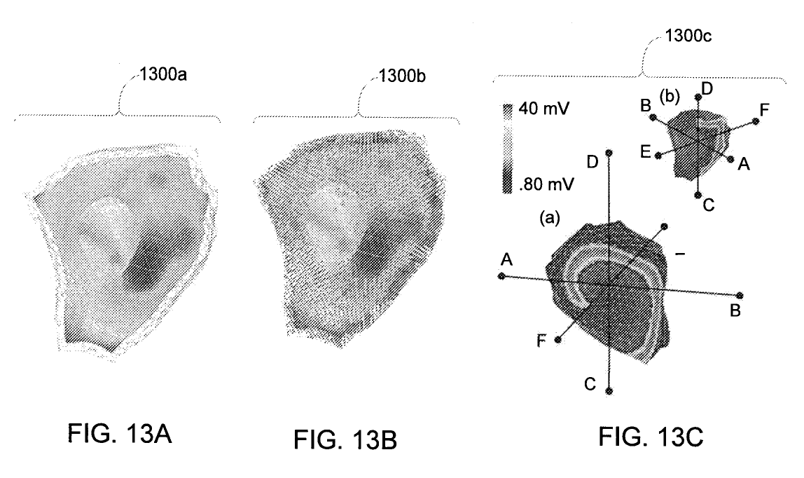

FIGS. 13A-C depict construction of a mathematical model of electrical activity using geometry of a left atria of a heart chamber as reconstructed by the surface reconstruction module 122. The original surface is illustrated as surface 1300a. The surface reconstruction module 122 fills the volume between the 3D rectangular mesh surfaces to form the 3D heart 1300b. The quantitative analysis module 126 simulates a reentry wave in a prepared model 1300c. The quantitative analysis module 126 can utilize electrical activity information (e.g., voltage, conduction velocity, etc.) to create a mathematical model of the body component. In other examples, the quantitative analysis module 126 simulates a model of arrhythmias based on different types of data and/or information (e.g., graphical reports, text reports, virtual ablation lines, etc.).

The reconstructed geometry of the heart chamber 1300b can be utilized to construct the numerical model 1300c of heart electrical activity. The quantitative analysis module 126 can construct the model utilizing the following steps:
1. The reconstructed surface is copied. The 3D position of the nodes of the copied surface is shifted so that each node is in defined distance outward (e.g., two mm, five mm, etc.) from corresponding node in original surface. This results in two surfaces separated by defined distance.
2. The volume between the original surface and the copied surface is filled with a 3D rectangular mesh of simulation nodes locally connected as illustrated in FIG. 13B.
3. Each simulation node stores time dependent variables describing its electrophysiological state and its dynamics is described using a mathematical model (e.g., FitzHugh-Nagumo model).

The above-described apparatuses, systems, and methods can be implemented in digital electronic circuitry, in computer hardware, firmware, and/or software. The implementation can be as a computer program product (i.e., a computer program tangibly embodied in an information carrier). The implementation can, for example, be in a machine-readable storage device and/or in a propagated signal, for execution by, or to control the operation of, data processing apparatus. The implementation can, for example, be a programmable processor, a computer, and/or multiple computers.

A computer program can be written in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the invention by operating on input data and generating output. Method steps can also be performed by and an apparatus can be implemented as special purpose logic circuitry. The circuitry can, for example, be a FPGA (field programmable gate array) and/or an ASIC (application-specific integrated circuit). Modules, subroutines, and software agents can refer to portions of the computer program, the processor, the special circuitry, software, and/or hardware that implements that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can include, can be operatively coupled to receive data from and/or transfer data to one or more mass storage devices for storing data (e.g., magnetic, magneto-optical disks, or optical disks).

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

To provide for interaction with a user, the above described techniques can be implemented on a computer having a display device. The display device can, for example, be a cathode ray tube (CRT) and/or a liquid crystal display (LCD) monitor. The interaction with a user can, for example, be a display of information to the user and a keyboard and a pointing device (e.g., a mouse or a trackball) by which the user can provide input to the computer (e.g., interact with a user interface element). Other kinds of devices can be used to provide for interaction with a user. Other devices can, for example, be feedback provided to the user in any form of sensory feedback (e.g., visual feedback, auditory feedback, of tactile feedback). Input from the user can, for example, be received in any form, including acoustic, speech, and/or tactile input.

The above described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network). Examples of communication networks include a local area network (LAN), a wide area network (WAN), the Internet, wired networks, and/or wireless networks.

The system can include clients and servers. A client and a server are generally remote from each other and typically interact through a communication network. The relationship of client and server arises by virtue of computer programs running on the respective computers and having a client-server relationship to each other.

Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, bluetooth, code-division multiple access (CDMA) network, time division multiple access (TDMA) network, global system for mobile communications (GSM) network), and/or other circuit-based networks.

The transmitting device can include, for example, a computer, a computer with a browser device, a telephone, an IP phone, a mobile device (e.g., cellular phone, personal digital assistant (PDA) device, laptop computer, electronic mail device), and/or other communication devices. The browser device includes, for example, a computer (e.g., desktop computer, laptop computer) with a world wide web browser (e.g., Microsoft® Internet Explorer® available from Microsoft Corporation, Mozilla® Firefox available from Mozilla Corporation). The mobile computing device includes, for example, a personal digital assistant (PDA).

Comprise, include, and/or plural forms of each are open ended and include the listed parts and can include additional parts that are not listed. And/or is open ended and includes one or more of the listed parts and combinations of the listed parts.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

What is claimed is:

1. A method for quantitative analysis of the distribution of electrophysiological parameters on a body component comprising:
reconstructing a shape of the body component based on coordinates associated with a plurality of data points;
interpolating the plurality of data points to form a value distribution map corresponding to the shape of the body component; and
generating a textual report and/or a graphical report based on the plurality of data points and/or the value distribution map.

2. The method of claim 1, wherein the data points comprise electrophysiological data points.

3. The method of claim 1, wherein the value distribution map comprises a 3-dimensional map of spatiotemporal distribution of values associated with the plurality of data points.

4. The method of claim 1, further comprising determining the coordinates associated with the data points based on electroanatomical information.

5. The method of claim 1, further comprising receiving the plurality of data points from a body component sensor.

6. The method of claim 1, further comprising transmitting the textual report and/or the graphical report to a computing device.

7. The method of claim 1, further comprising displaying the textual report and/or the graphical report on a display of a computing device.

8. The method of claim 1, wherein the body component comprises a heart, a lung, a liver, a stomach, a muscle, an organ, a tissue, or any combination thereof.

9. The method of claim 8, further comprising identifying one or more segments of damaged heart muscle areas in the heart associated with a health risk.

10. The method of claim 1, wherein the coordinates of data points are in 3-dimensional space.

11. The method of claim 1, further comprising modifying the value distribution map based on information associated with the body component sensor.

12. The method of claim 11, wherein the information associated with the body component sensor comprises an electrical potential of the body component.

13. The method of claim 1, wherein the value distribution map comprises a distribution of location activation time map, an electrical viability map, a conduction velocity map, a dominant frequency map, an activation regularity index map, a conduction phase map, an arrhythmogenesis map, or any combination thereof.

14. The method of claim 1, further comprising determining inhomogeneity of the body component based on a set of the plurality of data points.

15. The method of claim 1, further comprising determining homogeneity of the body component based on statistical properties of a conduction phase map.

16. The method of claim 1, further comprising:
determining a conduction phase map based on a conduction heterogeneity index; and
determining homogeneity of the body component based on the conduction phase map.

17. The method of claim 1, further comprising determining homogeneity of the body component based on minimum, maximum, mean, and/or standard deviation of a set of the plurality of data points and/or the value distribution map.

18. The method of claim 17, wherein the body component comprises a heart muscle and the set of the plurality of data points are associated with one or more ventricles and/or an atria of the heart muscle.

19. The method of claim 18, further comprising determining a quantifiable risk associated with arrhythmia of the heart muscle, the quantifiable risk being associated with inhomogeneity of the heart muscle and the inhomogeneity being associated with hypertrophic, cardiomyopathy; dilated cardiomyopathy; right ventricle arrhythmogenic cardiomyopathy; ischemic cardiomyopathy; after stem cells implantation in the heart muscle; a genetically disorder; or any combination thereof.

20. The method of claim 1, further comprising automatically dividing the shape of the body component into a plurality of segments.

21. The method of claim 20, further comprising generating the textual report and/or the graphical report for each segment in the plurality of segments based on the value distribution map associated with the segment.

22. The method of claim 20, further comprising automatically dividing the shape of the body component into the plurality of segments based on anatomical information associated with the body component.

23. The method of claim 20, further comprising simultaneously analyzing and comparing, in each segment in the plurality of segments, an area, a circumference, and/or the data points associated with the plurality of segments.

24. The method of claim 20, further comprising determining a segment in the plurality of segments to guide a treatment and/or diagnostic procedure.

25. The method of claim 24, further comprising:
diagnosing heart electrical activity; and
identifying a type and one or more characteristics of an arrhythmia based on the heart electrical activity.

26. The method of claim 24, further comprising guiding an ablation procedure based on an arrhythmogenic effect of the segment.

27. The method of claim 24, further comprising assessing an improvement of a heart muscle after stem cells injection.

28. The method of claim 1, further comprising determining a segment associated with the body component based on a relationship between a set of the plurality of data points and/or the value distribution map, the set of the plurality of data points being associated with electrophysiological information.

29. The method of claim 28, wherein the segment associated with the body component being an arrhythmogenic area of the body component and the method further comprising determining a geometrical location and spatial distribution of the segment on a surface of the body component.

30. The method of claim 28, wherein the arrhythmogenic area being associated with an area with low correlation, an area with positive correlation, an area with negative correlation, an area with abrupt change of correlation type, or any combination thereof.

31. The method of claim 30, further comprising determining a correlation utilizing a Spearman correlation and/or Pearson correlation.

32. The method of claim 28, wherein the relationship between the set of the plurality of data points being associated with a potential ablation target.

33. The method of claim 1, further comprising simulating a numerical model of arrhythmias associated with the body component based on the graphical report, the model comprising virtual ablation lines associated with the body component.

34. The method of claim 1, further comprising determining a quantifiable risk associated with arrhythmia associated with a geometrical feature of the body component, the geometrical feature being associated with a defined voltage and/or a defined conduction velocity.

35. The method of claim 1, further comprising determining the data points associated with a line, the line being designated based on user parameters and/or geometrical information associated with the body component.

36. The method of claim 35, further comprising:
determining an electrophysiological characteristic of the line; and
displaying the electrophysiological characteristic which enables a localization of an area associated with arrhythmogenesis.

37. A computer program product, tangibly embodied in an information carrier, the computer program product including instructions being operable to cause a data processing apparatus to:
reconstruct a shape of the body component based on coordinates associated with a plurality of data points;
interpolate the plurality of data points to form value distribution maps corresponding to the shape of the body component; and
generate a textual report and/or a graphical report based on the plurality of data points and/or the value distribution map.

38. An apparatus for quantitative analysis of the distribution of electrophysiological parameters on a body component, the apparatus comprising:
a surface reconstruction module for reconstructing a shape of the body component based on coordinates associated with a plurality of data points;
a data interpolation module for interpolating the plurality of data points to form value distribution maps corresponding to the shape of the body component; and
a quantitative analysis module for generating a textual report and/or a graphical report based on the plurality of data points and/or the value distribution map.

39. The apparatus of claim 38, further comprising a surface segmentation module for automatically dividing the shape of the body component into a plurality of segments.

40. The apparatus of claim 38, further comprising a map calculation module for determining a segment associated with the body component based on a relationship between a set of the plurality of data points, the set of the plurality of data points being associated with electrophysiological information.

41. The apparatus of claim 38, further comprising a 3-dimensional electroanatomical mapping module for determining the coordinates associated with the data points based on electroanatomical information.

42. An apparatus for quantitative analysis of the distribution of electrophysiological parameters on a body component, the apparatus comprising:
means for reconstructing a shape of the body component based on coordinates associated with a plurality of data points;
means for interpolating the plurality of data points to form value distribution maps corresponding to the shape of the body component; and
means for generating a textual report and/or a graphical report based on the plurality of data points and/or the value distribution map.
